(19) United States
(12) Patent Application Publication
(10) Pub. No.: US 2009/0240269 A1
(43) Pub. Date: Sep. 24, 2009

(54) Gastric Belt Preformed in a "c" Shape

(75) Inventor: Pierre-Andre Denis, Villeurbanne (FR)

Correspondence Address: DOWELL & DOWELL P.C., 103 Oronoco St., Suite 220, Alexandria, VA, 22314, US

(73) Assignee: Denis Medical

(21) Appl. No.: 12/309,023

(22) PCT Filed: Jul. 5, 2007
(86) PCT No.: PCT/FR2007/001139

§ 371 Date: Jan. 2, 2009

(30) Foreign Application Priority Data
Jul. 5, 2006 (FR) 0606117
Oct. 30, 2006 (FR) 0609494

(51) Int. Cl. A61B 17/122

(52) U.S. Cl. 606/157

(57) ABSTRACT

The invention relates to an inflatable gastric belt comprising an elongate tubular body (2), connecting means (10, 11) arranged in relation with the two ends (12, 19), and an inflation catheter. According to the invention, the tubular body (2) is preformed in such a way that, at rest and before closure of the belt, it has a longitudinal cross section with the general shape of a “C”.

The invention relates to the technical field of devices which are to be implanted at the junction between the abdominal part of the oesophagus and the stomach, in order to create a local restriction allowing the amount of food ingested by the patient wearing the device to be controlled.

In order to provide that local restriction it is known to employ an inflatable gastric belt or ring as described, for example, in patent applications EP 0 769 282, FR 2 799 118.

According to those documents, the inflatable gastric belt comprises an elongated flat tubular body, made of flexible material, which, at least in part, is resiliently deformable and which defines a tight chamber which can be inflated to exhibit a working face, which is to be placed in contact with the stomach, and a back on the opposite side from the working face.

In order to allow the belt to be closed to form a ring, the belt also comprises connecting means which are provided at the ends of the tubular body and allow the gastric belt to be closed in the form of a ring, the working face being oriented, of course, towards the inside. Finally, the gastric belt also comprises an inflation catheter which is connected tightly to the inflatable chamber and is to be connected to inflation means. The inflation means can be constituted, for example, by a housing provided with a self-closing membrane which can be pierced by a syringe needle or the like, by means of which it is possible to effect an injection or a removal of fluid, such as, for example but not necessarily, physiological serum, in order to control the inflation of the chamber and thus the dimensions of the gastric constriction produced by means of the inflatable gastric belt.

However, although such bands have generally been satisfactory and have in most cases allowed the desired therapeutic effect to be achieved after implantation, it has been found that their implantation time can be relatively long owing to difficulties with closing the gastric belt, especially during fitting by coelioscopy.

There is therefore a need for a novel type of gastric belt which is simpler to implant and, especially, to close during fitting by coelioscopy.

In order to achieve that object, the invention relates to an inflatable gastric belt comprising:
an elongated tubular body made of flexible material which, at least in part, is resiliently deformable and which defines a tight inflatable chamber and has a back and a working face,
connecting means which are arranged at the two ends of the tubular body and allow the gastric belt to be closed in the form of a ring, the working face being arranged on the inside of the ring,
and an inflation catheter which is to be connected to inflation means and is connected tightly to the inflatable chamber via an inflation orifice provided in the back of the body or at one end of the elongated tubular body.

According to the invention, the inflatable gastric belt is characterized in that the elongated tubular body is preformed so that, at rest and before the belt is closed, it has a generally “C”-shaped longitudinal cross-section.

The fact that the tubular body is preformed in a “C” shape substantially facilitates the fitting and closing of the gastric belt, especially during implantation by coelioscopy, in that it is no longer necessary to hold the two ends of the tubular body in order to start forming the ring shape.

According to a feature of the invention, the tubular body extends, at rest and before the belt is closed, over an angular sector of from 160° to 300°, preferably from 250° to 280°. It has been found that those values are particularly suited to the anatomical characteristics of the part of the stomach in which the gastric belt is implanted and greatly facilitate the operation of closing the belt. The fact that the belt is preformed avoids errors when fitting the band behind the stomach because, given that the belt is preformed in a C shape, it is impossible to close the belt with the working face on the outside.

In order to adapt the gastric belt to the implantation conditions in the best possible manner, according to another feature of the invention the working face of the inflatable tubular body has, at rest and before the belt is closed, a radius of curvature of from 22 mm to 35 mm, preferably from 22 mm to 30 mm, more particularly preferably from 22 mm to 29 mm.

Still within the scope of a better adaptation to the anatomical constraints of the implantation site, according to yet another feature of the invention the working face of the inflatable tubular body has an extended length of from 110 mm to 130 mm, preferably from 112 mm to 117 mm or from 123 mm to 127 mm.

According to another feature of the invention, which aims to provide better stability of the gastric belt on the part of the stomach that it surrounds, the inflatable tubular body has, at rest and before the belt is closed, in a central region, a rectangular or oblong cross-section. Accordingly, at rest and before inflation, the working face of the inflatable tubular body has the shape of a portion of a cylindrical ring.

According to the invention, the connecting means which allow the belt to be closed to form a ring can be produced in any appropriate manner. In a preferred but not exclusive embodiment of the gastric belt according to the invention, the connecting means comprise on the one hand, in the region of a first end of the body, called the inflation end, a tail-piece for connecting the catheter to the inflatable chamber, and on the other hand, close to the opposite end, called the head end, at least one arched member provided on the back of the body for the passage of the connecting tail-piece.

Within the scope of that preferred embodiment of the connecting means, and in order to further facilitate the fitting and closing of the belt to form a ring, at least a portion of the connecting tail-piece has, at rest and before the belt is closed, a curved shape which extends as a continuation of the curvature of the tubular body.

In a preferred embodiment, in order to facilitate closure in the best possible manner, the curved portion of the connecting tail-piece extends over an angular sector of from 45° to 75°, preferably from 50° to 60°.

In order to facilitate gripping of the connecting tail-piece and of the catheter connected thereto, the connecting tail-piece comprises a proximal curved portion and a distal straight portion which extends as a continuation of the curved portion in a direction at a tangent to the curvature of the curved portion.

According to yet another feature of the invention, and in order to reduce the risks of the gastric belt becoming caught in the tissues surrounding the stomach, the edges of the tubular body connecting the inflation end to the connecting tail-piece have a flattened “S” shape in order to define a connecting zone that does not have a projecting angle.

According to the invention, the gastric belt can be locked in the ring shape in various ways. Within the scope of the preferred embodiment of the gastric belt, and in order to effect locking, the connecting tail-piece has at least one locking arrangement in the shape of a fir tree comprising two generally “V”-shaped returns which are arranged on each side of the connecting tail-piece while being oriented towards the tubular body and the two main sides of which form an angle less than or equal to 45°. The presence of the two V-shaped returns confers on each fir-tree-shaped arrangement an arrow or lanceolated shape.

In a preferred but not exclusive embodiment of the invention, the gastric belt comprises at least two locking arrangements in the shape of a fir tree, a first, so-called proximal locking arrangement, which extends over the proximal curved portion of the connecting tail-piece, and a second, so-called distal locking arrangement, which extends over the distal straight portion of the connecting tail-piece.

In order to complete locking and prevent the V-shaped returns from becoming caught on the tissues surrounding the part of the stomach in which the gastric belt is implanted, the belt preferably comprises, in the region of the back of the tubular body, a number of arched members equal to the number of fir-tree-shaped locking arrangements on the connecting tail-piece, each arched member comprising two notches or two webs for immobilizing the “V”-shaped returns of the corresponding locking arrangement.

Still in order to ensure a better performance of the locking of the connecting means, and according to another feature of the invention, each arched member defines a tunnel for receiving the connecting tail-piece, the inside wall of the tunnel converging in a direction away from the head of the tubular body, and the portion of the connecting tail-piece that is to be placed in the tunnel defined by the arched member when the belt is in the closed position has a complementary shape. As has been stated hereinbefore, the shape complementarity strengthens the locking and additionally prevents tissues from developing between the connecting tail-piece and the arched member or members straddling it.

According to the invention, the inflatable tubular belt can be manufactured in various ways. In a preferred embodiment, the tubular body and the connecting tail-piece form a one-piece assembly obtained by injection moulding in an arc-shaped cavity. It will then be possible for the free end of the body to be closed off or formed by an added plug.

According to another feature of the invention, which aims to facilitate the location of the gastric belt by X-ray radiography, the belt comprises at least one thread made of radio-opaque material which is integrated in a wall of the inflatable tubular body. The thread of opaque material may be incorporated on its own into one or other of the walls of the inflatable tubular body or it may be incorporated into an inextensible flexible reinforcing band which is integrated in the back of the inflatable tubular body.

According to yet another feature of the invention, and in order to ensure optimum visibility of the radio-opaque thread, the latter will be made of a radio-opaque material comprising gold.

According to the invention, the inflatable gastric belt can be made of any suitable biocompatible material, preferably of grey-coloured or matt biocompatible silicone so that it has an outer surface which does not dazzle when it is illuminated and observed by means of a coelioscopy device.

It will be appreciated that the various features of the invention mentioned above can be employed together or only in part, in different combinations in order to produce an inflatable gastric belt according to the invention.

In addition, various other features of the invention will become apparent from the description below, which is given with reference to the accompanying drawings which show a preferred but non-limiting embodiment of an inflatable gastric belt according to the invention.

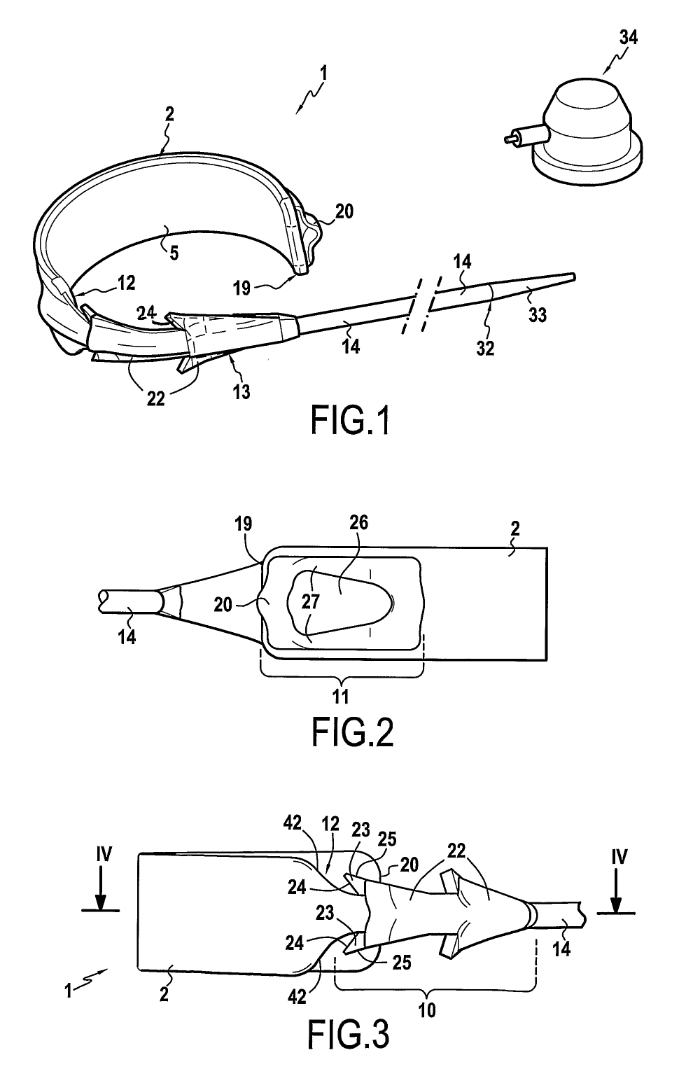

FIG. 1 is a perspective view, in a rest state, of a gastric belt according to the invention.

FIG. 2 is a side view from the head end of the inflatable tubular body of the gastric belt shown in FIG. 1.

FIG. 3 is a side view from the connecting end of the inflatable tubular body of the gastric belt shown in FIG. 1.

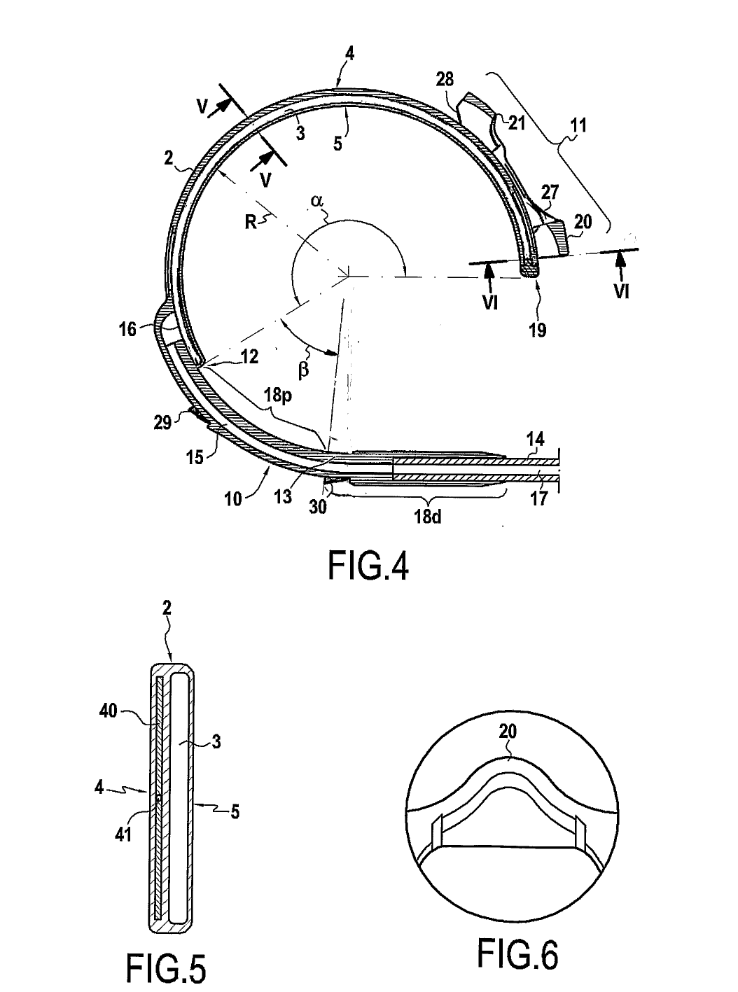

FIG. 4 is a longitudinal section according to plane IV-IV of FIG. 3 of the gastric belt.

FIG. 5 is a transverse section according to line V-V of FIG. 4.

FIG. 6 is a detailed view of an arched member of the gastric belt according to line VI-VI of FIG. 2.

A gastric belt according to the invention, shown in FIGS. 1 to 4 and designated as a whole by reference numeral 1, comprises a tubular body 2 which defines at least one tight inflatable chamber 3. According to the invention, the body 2, in elevation when viewed from the top, is generally C-shaped.

It must additionally be noted that, when at rest, that is to say when the chamber 3 is in the deflated state and before the belt is closed to form a ring, the body 2 is generally flat in shape and has a substantially rectangular or oblong transverse cross-section, as is shown more particularly in FIG. 5, the inflatable chamber 3 having a transverse cross-section whose general shape is likewise inscribed in a rectangle.

The gastric belt 1 has a back 4 and a working face 5 which is to come into contact with the region of the oesophagus or stomach in which the belt will be placed, as will be seen hereinbelow.

The general C shape of the inflatable tubular body 2 confers on the longitudinal cross-section of the body 2, as is shown more particularly in FIG. 4, a C shape which extends over an angular sector whose angle at the centre a has a value of from 160° to 300°, preferably from 250° to 280°, it being possible for the angle a also to have as its value one or other of the limits of those various ranges. At rest and before the belt is closed to form a ring, the working face 5 of the body 2 then has the shape of a portion of a cylindrical ring having a constant radius of curvature R of from 22 mm to 35 mm, preferably from 22 mm to 30 mm, more particularly preferably from 22 mm to 29 mm, it being possible for the radius of curvature R also to have as its value one or other of the limits of those various ranges. In addition, the working face of the inflatable tubular body 2 has an extended length of from 110 mm to 130 mm, preferably from 112 mm to 117 mm or from 123 mm to 127 mm, the last two ranges corresponding to two separate sizes of gastric belt according to the invention.

In order to allow the belt 1 to be closed to form a ring, the belt 1 also comprises connecting means 10 and 11 provided at the two ends of the belt. The connecting means 10 and 11 can be produced in any appropriate manner.

According to the example shown, the connecting means 10 first comprise, in the region of a first end 12 of the body 2, called the inflation end, a tail-piece 13 for connecting a catheter 14 to the inflatable chamber 3.

According to the example shown, the connecting tail-piece 13 extends substantially as a longitudinal continuation of the body 2 and has an inner channel 15 which is connected to the chamber 3 by an inflation orifice 16, as is shown more particularly in FIG. 5. It will be appreciated that the channel 15 communicates with the inner conduit 17 of the catheter 14, which is then connected to the chamber 3, via the inflation orifice 16. The connecting tail-piece 13 has, at rest and before the belt is closed, a curved shape which extends as a continuation of the curvature of the body 2. The curved shape can extend over all or part of the length of the connecting tail-piece 13. According to the example shown, the curved shape affects only a portion 18p, called the proximal portion, of the connecting tail-piece, which extends between the inflation end 12 and a central region of the connecting tail-piece 13. The curved proximal portion 18p extends over an angular sector whose angle at the centre β is from 45° to 75°, preferably from 50° ° to 60°, it being possible for the angle β also to have as its value one or other of the limits of those various ranges. According to the example shown, the proximal portion 18p is extended by a straight distal portion 18d which extends in a direction at a tangent to the curvature of the curved portion 18p.

In addition, the connecting means 11, which are located in the region of or close to the end that is opposite the inflation end 12, which is called the free end 19, are constituted in the example shown by at least one and, in the present case, by two arched members 20, 21 which are arranged in the region of the back 4 of the body 2 and are to receive the tail-piece 13.

According to the example shown, the two arched members 20, 21 are located at a distance from one another, and the tail-piece 13 comprises catch or anti-return means 22 which are to prevent the connecting tail-piece 13 from being inadvertently withdrawn once it has been engaged in the arched members 20, 21. According to the example shown, the anti-return catch means comprise two fir-tree-shaped proximal and distal arrangements 22, each of which is to cooperate with a corresponding arched member 20, 21. In a preferred manner, the first locking arrangement, or locking arrangement, is provided on the so-called proximal curved portion 18p of the connecting tail-piece, while the second locking arrangement, or distal locking arrangement, extends over the distal portion 18d of the connecting tail-piece. Each locking arrangement 22 comprises two generally V-shaped returns 23 which are arranged on each side of the connecting tail-piece while being oriented towards the tubular body 2. As will be seen in FIG. 1, the returns 23 are arranged so that they are substantially parallel or at a tangent to the cylinder generating the surface of the working face 5 of the inflatable tubular body 2. The main sides 24, 25 of the returns 23, which confer thereon their V shape, form an angle less than or equal to 45°.

In order to complete the locking, the first arched member 20, or proximal arched member, has wings which extend towards the second arched member 21 in such a manner as to define a cavity 26 for receiving all or part of the locking arrangement 22 closest to the end 12 of the body 2, called the proximal end. The arched member 20 further comprises two webs beneath which the returns 23 of the proximal locking arrangement 22 located in the cavity are engaged. In the same manner, the second arched member 21 comprises, in the region of its limbs for connection with the back 4 of the body 2, two notches 28 in which the returns 23 of the second locking arrangement engage when the gastric belt 1 is closed to form a ring. As is shown in FIG. 4, the walls of each notch 28 form an acute angle with the back 4 of the belt. The surface defining the main inside face 24 of each return 23 forms, with the lower face of the locking arrangement, an obtuse angle that is complementary to the acute angle of the notches 28, as is shown in FIG. 1. This shape complementarity ensures effective locking.

Still within the scope of better locking, each arched member 20, 21 defines a tunnel for receiving the connecting tail-piece 13, the inside wall of which converges in a direction away from the head 19 of the body 2, as is shown more particularly in FIGS. 1 and 6. The parts 29 and 30 of the connecting tail-piece 13 which are to be placed in each of the tunnels defined by the arched members 20 and 21 when the belt is in the closed position to form a ring, have a complementary shape so that on the one hand they occupy the whole of the inside space of the corresponding arched member and on the other hand they prevent the tail-piece 13 from slipping and reducing the inside diameter of the gastric belt 1 that has been closed to form a ring. The fir-tree-shaped arrangements 22 in turn prevent the locking tail-piece 13 from slipping in the opposite direction.

In addition, the free end 32 of the catheter 14 is closed off by a conical plug 33, which prevents material from being introduced into the channel of the catheter and facilitates the introduction of the catheter into the arched member 20 during closure of the belt.

The plug 33 will be cut after the belt has been closed, for the fitting of the inflation means 34.

The inflatable gastric belt 1 can be made of any suitable biocompatible material, such as, for example, biocompatible or implantable-grade silicone, which confers on the body 2 the flexibility and elasticity required for inflation of the chamber 3. Preferably, the biocompatible silicone used will be coloured so that it has a grey colour and its outside surface does not dazzle when it is illuminated and observed by means of a coelioscopy device. The non-dazzling nature can also be obtained by conferring a matt appearance on the outside surface of the gastric belt 1.

The connecting means 10, 11 are then operated in order to close the inflatable belt 1 to form a ring, as is shown in FIG. 4.

In addition, according to another feature of the invention, in order to ensure better control of the gastric restriction formed by the belt 1, it is also provided to incorporate, in the wall of the body 2 constituting the back 4, a flexible inextensible reinforcing band 40. Accordingly, when the chamber 3 is inflated, the belt 1 undergoes a substantially centripetal deformation. The flexible reinforcing band 40 can be made of any suitable inextensible flexible material, such as, for example but not exclusively, a Dacron fabric.

Preferably, the reinforcement 40 is completely embedded in the wall of the body 2 forming the back 4 and is completely surrounded by the material constituting the body 2.

In addition, in order to render the gastric belt 1 according to the invention easily detectable by X-rays, a radio-opaque thread 41, such as a gold thread, is incorporated into body 2 and preferably into the back thereof. The radio-opaque thread 41 can either be independent of the reinforcing band 40 or can form an integral part thereof.

According to a feature of the invention, the edges 42 of the tubular body 2 connecting the inflation end 12 to the connecting tail-piece have a flattened “S” shape so as to define a connecting zone that does not have a projecting angle. That shape of the connecting region between the body 2 and the connecting tail-piece 13 facilitates the penetration of the gastric belt into the tissues and/or the adipose masses surrounding the stomach during implantation of the gastric belt according to the invention.

It will be appreciated that various modifications can be made to the invention within the scope of the claims.

1-19. (canceled)

20. Inflatable gastric belt comprising:
an elongated tubular body (2) made of flexible material which, at least in part, is resiliently deformable and which defines a tight inflatable chamber (3) and has a back (4) and a working face (5), connecting means (10, 11) which are arranged at the two ends (12, 19) of the tubular body (2) and allow the gastric belt to be closed in the form of a ring, the working face (5) being arranged on the inside of the ring, and an inflation catheter (14) which is to be connected to inflation means (34) and is connected tightly to the inflatable chamber (3) via an inflation orifice (16) provided in the back (4) or at one end of the elongated tubular body (2), wherein, at rest and before the belt is closed, the tubular body (2) has a generally flat shape with a substantially rectangular or oblong transverse cross-section and is preformed so that it has a generally “C”-shaped longitudinal cross-section.

21. Inflatable gastric belt according to claim 20, wherein the tubular body (2) extends, at rest and before the belt is closed, over an angular sector (a) of from 160° to 300°, preferably from 250° to 280°.

22. Inflatable gastric belt according to claim 20, wherein the working face (5) of the tubular body (2) has, at rest and before the belt is closed, a radius of curvature (R) of from 22 mm to 35 mm, preferably from 22 mm to 30 mm, more particularly preferably from 22 mm to 29 mm.

23. Inflatable gastric belt according to claim 20, wherein the working face (5) of the tubular body (2) has an extended length of from 110 mm to 130 mm, preferably from 112 mm to 117 mm or from 123 mm to 127 mm.

24. Inflatable gastric belt according to claim 20, wherein the connecting means (10, 11) comprise on the one hand, in the region of a first end (12) of the body, called the inflation end, a tail-piece (13) for connecting the catheter (14) to the inflatable chamber, and on the other hand, close to the opposite end (19), called the head end, at least one arched member (20, 21) provided on the back of the body for the passage of the connecting tail-piece.

25. Inflatable gastric belt according to claim 24, wherein at least a portion (18p) of the connecting tail-piece (13) has, at rest and before the belt is closed, a curved shape as a continuation of the curvature of the tubular body.

26. Inflatable gastric belt according to claim 25, wherein the curved portion (18p) of the connecting tail-piece (13) extends over an angular sector (b) of from 45° to 75°, preferably from 50° to 60°.

27. Inflatable gastric belt according to claim 25, wherein the connecting tail-piece (13) comprises a proximal curved portion (18p) and a distal straight portion (18d) which extends as a continuation of the curved portion (18p) in a direction at a tangent to the curvature of the curved portion (18p).

28. Inflatable gastric belt according to claim 24, wherein the edges of the tubular body connecting the inflation end to the connecting tail-piece have a flattened “S” shape so as to define a connecting zone that does not have a projecting angle.

29. Inflatable gastric belt according to claim 24, wherein the connecting tail-piece (13) has at least one locking arrangement (22) in the shape of a fir tree comprising two generally “V”-shaped returns (23) which are arranged on each side of the connecting tail-piece (13) while being oriented towards the tubular body (2) and the two main sides (21, 25) of which form an angle less than or equal to 45°.

30. Inflatable gastric belt according to claim 29, wherein it comprises at least two locking arrangements in the shape of a fir tree, a first, so-called proximal locking arrangement, which extends over the proximal curved portion of the connecting tail-piece, and a second, so-called distal locking arrangement, which extends over the distal straight portion of the connecting tail-piece.

31. Inflatable gastric belt according to claim 29, wherein it comprises, in the region of the back (4) of the tubular body (2), a number of arched members (20, 21) equal to the number of fir-tree-shaped locking arrangements (22) on the connecting tail-piece (13), each arched member comprising two notches (28) or two webs (27) for immobilizing the “V”-shaped returns of the corresponding locking arrangement.

32. Inflatable gastric belt according to claim 24, wherein each arched member (20, 21) defines a tunnel for receiving the connecting tail-piece, the inside wall of the tunnel converging in a direction away from the head (19) of the tubular body, and in that the portion (29, 30) of the connecting tail-piece that is to be placed in the tunnel defined by the arched member when the belt is in the closed position has a shape complementary to that of the tunnel.

33. Inflatable gastric belt according to claim 20, wherein the tubular body (2) and the connecting tail-piece (13) form a one-piece assembly obtained by injection moulding in an arc-shaped cavity.

34. Inflatable gastric belt according to claim 20, wherein it comprises at least one thread (41) made of radio-opaque material integrated in a wall of the tubular body (2).

35. Inflatable gastric belt according to claim 34, wherein the thread of radio-opaque material (41) is incorporated into an inextensible flexible reinforcing band (40) which is integrated in the back (4) of the tubular body (2).

36. Inflatable gastric belt according to claim 34, wherein the thread of radio-opaque material comprises gold.

37. Inflatable gastric belt according to claim 20, wherein it is made of grey-coloured or matt biocompatible silicone so that it has a non-dazzling outer surface.

38. Inflatable gastric belt according to claim 27, wherein it comprises at least two locking arrangements in the shape of a fir tree, a first, so-called proximal locking arrangement, which extends over the proximal curved portion of the connecting tail-piece, and a second, so-called distal locking arrangement, which extends over the distal straight portion of the connecting tail-piece.
